(19) United States
(12) Patent Application Publication
(10) Pub. No.: US 2010/0063304 A1
(43) Pub. Date: Mar. 11, 2010

(54) APPARATUS FOR AUTOMATIC CATALYST EXCHANGE IN A REACTOR WITH A BUNDLE OF CATALYST TUBES

(75) Inventors: Gerhard Olbert, Dossenheim (DE); Ralf Bihl, Monzernheim (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/543,816

(22) Filed: Aug. 19, 2009

(30) Foreign Application Priority Data
Sep. 9, 2008 (EP) 08163957.7

(51) Int. Cl. C07D 307/89; C07C 45/00; B01J 8/04; C07C 53/00; C07D 307/36; C07D 303/00; B21D 51/16

(52) U.S. Cl. 549/247; 568/449; 422/197; 562/512; 549/262; 549/513; 29/890

(57) ABSTRACT

What is proposed is an apparatus for automatic catalyst exchange in a plurality of process steps in the catalyst tubes of a reactor with a bundle of catalyst tubes, which are welded in or rolled in between an upper tube plate and a lower tube plate in longitudinal direction of the reactor, and welded tight,

comprising a tool carrier device, which is arranged so as to be mobile in a plane above and parallel to the upper tube plate, and which can be equipped with an exchangeable tool for each process step, the exchangeable tool being mobile together with the tool carrier device in the plane above and parallel to the upper tube plate and also vertically, in the direction of the longitudinal axis of the reactor, and being able to access each individual catalyst tube with control by means of a control unit, the position of the longitudinal axis of each individual catalyst tube being measured or calculated and recorded in the control unit in a position database.

The invention relates to an apparatus and to a process for automatic catalyst exchange in the catalyst tubes of a reactor with a bundle of catalyst tubes, and to a use thereof.

In chemical process technology, reactions are frequently carried out in the presence of heterogeneous catalysts which are introduced into catalyst tubes in beds in the form of shaped catalyst bodies. In this case, a multitude of catalyst tubes is arranged in parallel to one another, in the longitudinal direction of the reactor, frequently up to 30 000 or even up to 40 000 catalyst tubes.

The steps associated with catalyst exchange, i.e. preparation of the catalyst tubes to accommodate new shaped catalyst bodies used for the first time, filling of the catalyst tubes with the shaped catalyst bodies and if appropriate with inert material, checking of the fill height and pressure drop in the individual catalyst tubes, emptying of the tubes of spent shaped catalyst bodies and retesting the catalyst tubes before refilling, have to date been carried out in a manual and correspondingly time-consuming and fault-prone manner.

This procedure is additionally associated with exposure of personnel to harmful substances, the reactor shutdown times are generally high and the availability of the reactor is correspondingly reduced. The sources of error in the case of manual filling of the tubes are unduly high, especially as far as the homogeneous filling of the tubes is concerned, and there is the risk of individual tubes remaining unfilled. In spite of very strict checking, it occurs time and again in tubular reactors with a large number of catalyst tubes that individual catalyst tubes are not filled as specified.

It was therefore an object of the invention to provide an improved process for catalyst exchange in the catalyst tubes of a tube bundle reactor, which does not have the above disadvantages. More particularly, even in the case of large reactors with a multitude of catalyst tubes, frequently up to 30 000 or even up to 40 000 catalyst tubes, it should be possible for all tubes uniformly and in accordance with defined standards for accommodation of shaped catalyst bodies, to be checked, filled, emptied after use of the catalyst material, and cleaned and filled again. At the same time, more particularly, a more rapid catalyst exchange with shortened reactor shutdown times and correspondingly increased availability of the reactors shall be enabled, with a reduction in the labor intensity and the exposure of personnel to harmful substances, while ensuring documented 100% checking of the working steps.

This object is achieved in accordance with the invention by an apparatus for automatic catalyst exchange in a plurality of process steps in the catalyst tubes of a reactor with a bundle of catalyst tubes, which are welded in or rolled in between an upper tube plate and a lower tube plate in longitudinal direction of the reactor, and welded tight,

comprising a tool carrier device, which is arranged so as to be mobile in a plane above and parallel to the upper tube plate, and which can be equipped with an exchangeable tool for each process step, the exchangeable tool being mobile together with the tool carrier device in the plane above and parallel to the upper tube plate and also vertically, in the direction of the longitudinal axis of the reactor, and being able to access each individual catalyst tube with control by means of a control unit, the position of the longitudinal axis of each individual catalyst tube being measured or calculated and recorded in the control unit in a position database.

The invention is independent of the specific arrangement of the catalyst tubes over the reactor cross section: they may be arranged homogeneously over the reactor cross section, especially in a triangular pitch, or else in other pitches. It is possible that the catalyst tubes are arranged leaving an empty central interior in the reactor and if appropriate a space free of catalyst tubes at the inner jacket of the reactor, or else leaving two opposite spaces free of catalyst tubes at the inner jacket of the reactor.

The catalyst tubes are, as usual, arranged in longitudinal direction of the reactor and welded or rolled into an upper tube plate and a lower tube plate, and welded tight.

The inventive apparatus is suitable especially for automatic catalyst exchange in reactors having from 100 to 40 000 catalyst tubes, preferably having from 1500 to 40 000 catalyst tubes, or else having from 2500 to 40 000 catalyst tubes.

The invention also provides a process for automatic catalyst exchange in a plurality of process steps in the catalyst tubes of a reactor with a bundle of catalyst tubes, which are welded in or rolled in between an upper tube plate and a lower tube plate in longitudinal direction of the reactor, and welded tight,
the position of the longitudinal axis of each individual catalyst tube first being determined, especially by means of a 3D laser scanner, or calculated, and recorded in a position database of a control unit,
and then the control unit being used to access each individual catalyst tube of the bundle of catalyst tubes with a tool in each case to perform each individual process step for automatic catalyst exchange, the tool being arranged so as to be exchangeable on a tool carrier device which is arranged so as to be mobile in a plane above and parallel to the upper tube plate, and the tool together with the tool carrier device being moved in the plane above and parallel to the upper tube plate, and also vertically in the direction of the longitudinal axis of the reactor, while each individual process step for automatic catalyst exchange is performed in each case.

According to the invention, the exact positions of all catalyst tubes, specifically the positions of the longitudinal axes thereof, or, in other words, the positions of the tube centers of a cross-sectional plane at right angles to the longitudinal axis of the reactor, are first calculated or measured and recorded in a position database.

Determination by means of calculation is possible provided that the reactor has entirely homogeneously positioned catalyst tubes, especially in the case of reactors which have never been operated before.

For this purpose, for example, in the case of a tubular reactor with a catalyst tube bundle which leaves an empty space free of catalyst tubes both in the central region of the reactor and at the inner wall of the reactor, four relevant dimensions are required, specifically the internal diameter and the external diameter of the catalyst tube bundle, the hole separation, i.e. the center separation between two adjacent catalyst tubes, and the angle between the axes connecting the centers of three adjacent catalyst tubes.

These relevant dimensions are used, by means of a calculation program, to calculate the number and exact position of the catalyst tubes.

However, especially in the case of reactors which have already been operated, it may be necessary to determine the position of the individual catalyst tubes by measurements. For this purpose, it is possible to use especially 3D laser measuring systems, for example a laser scanner of the LMS 400® type from Sick AG. The 3D laser measurement system determines, with different angles of incidence (the eye sees a projected line), the laser beam length (distance and angle, i.e. two dimensions). The third dimension is obtained from the rotary encoder of the shaft(s) responsible for the movement of the scanner, the pulses from which are looped through the scanner and combined with angle and distance, and then transmitted as a data packet to a computer. These data are used to determine the positions of the individual catalyst tubes by means of image processing, which are then stored in a position database. The control unit of the tool carrier device takes the position coordinates from this position database and integrates them into the individual working steps.

Alternatively, it is also possible to use 2D or 3D camera systems. In the case of 2D images, the tube positions are calculated by means of image processing and the third dimension, i.e. the position of the reactor plate, is determined by means of distance measurement, for example with a measuring probe or a laser measuring system, by means of three measurement points. These data are used to obtain the 3D coordinates of the tubes which are recorded in a position database.

In 3D camera systems which work, for example, by the light section method, the tube positions are likewise calculated by means of image processing and the data are recorded in a position database.

The inventive apparatus comprises a tool carrier device which is arranged so as to be mobile within the interior of the reactor in a plane above and parallel to the upper tube plate. The tool carrier device is designed such that it can move over the entire cross-sectional area, in order that each individual catalyst tube can be accessed.

In one embodiment, the tool carrier device has all six degrees of freedom of motion.

In a preferred embodiment, the tool carrier device comprises a tool carrier arm. In this case, the tool carrier arm may especially be mounted at the center of the reactor and, by means of a servomotor, which is preferably positioned at the opposite end of the tool carrier arm, may be moved over the entire reactor cross section in a circular motion.

In a further embodiment, the tool carrier device comprises a slide.

In a further embodiment, the tool carrier device comprises a gantry crane.

The tool carrier device may especially be an autonomous, self-propelled robot which is controlled by a global or regional navigation system.

It is also possible to provide two or more, especially two, tool carrier arms simultaneously, which, for example, are moved in the same manner as hour and minute hands of a clock. These may be mounted in the reactor center axis either in a suspended or stationary manner.

It is possible for a variety of different tools required to perform the different process steps for automatic catalyst exchange to be secured exchangeably on the tool carrier device, and to be moved in the direction of the tool carrier arm and in the direction of the longitudinal axis of the reactor, more particularly by means of a servomotor.

In this case, it is possible to exchangeably secure a single tool for each of the different process steps on the tool carrier device; however, it is also possible to exchangeably arrange two or more tools for performing the same process step or different process steps simultaneously on the tool carrier device.

The tools are moved with control by means of a control unit which possesses the position of each individual catalyst tube from the position database.

Preference is given to documenting all process steps and to releasing the reactor only when the preset tolerance ranges have been complied with for all process steps.

The individual process steps for automatic catalyst exchange are:

first, when using new catalyst tubes for the first time, the first cleaning thereof, especially because the catalyst tubes may have corroded slightly. The first cleaning can preferably be effected by means of high-pressure cleaning, especially with water, or else mechanically, preferably by brushing.

To this end, the particular corresponding tools, i.e. more particularly, high-pressure cleaners or brushes, are arranged so as to be mobile on the tool carrier.

In a further process step, the material of the catalyst tubes is tested for material damage, as a so-called zero measurement. More particularly, there is a test of whether there is any damage to the catalyst tubes or any shrink holes, and whether the catalyst tubes have a homogeneous wall thickness. The material is tested, preferably by means of eddy current analysis; the corresponding tool which is arranged so as to be mobile on the tool carrier arm is an eddy current probe. It is also possible to test material by means of ultrasound.

The position of the tool for material testing can be used to determine the exact position of any and each site of damage.

The tool for material testing may also be a camera, i.e. the material test may be an endoscopic test, especially a videoscopic test.

In a further process step, there is a check as to whether the catalyst retaining devices at the lower end of the catalyst tubes, the so-called catalyst supports, are incorporated in each individual catalyst tube. The check can be effected especially by means of detectors for metallic objects, especially optical, inductive, capacitive or ultrasound sensors.

Preferably, especially after the catalyst retaining devices have already been mounted, all catalyst tubes are covered in all subsequent process steps, preferably by means of a stopper on each, apart from the catalyst tube on which a process step is currently being performed.

In a next process step, the catalyst tubes provided with catalyst retaining devices are filled with one or more layers of shaped catalyst bodies and if appropriate with one or more layers of inert material.

Preference is given to first introducing a first layer of inert material in a defined amount from a reservoir vessel. The amount released may be determined gravimetrically, preferably by means of a load cell with strain gauges. The target weight can be preset as desired by means of software.

It is also possible to regulate the amount of material released volumetrically by means of a supply tube with a defined volume. In this way, any desired release volume can be established by a simple change in the supply tube.

The catalyst and/or inert material is advantageously conveyed by means of a vacuum conveying system, automatically and “just in time” from an external reservoir vessel into the accompanying reservoir vessel on the tool carrier arm, such that the filling process need not be interrupted for material replenishment, since the accompanying material supply is effectively infinite.

After the first layer, preferably of inert material, has been introduced, the fill height is checked, especially by means of a laser.

Subsequently, in the same way, one or more layers of shaped catalyst bodies are metered in gravimetrically or volumetrically, and, after each layer, the fill height and, if appropriate, the pressure drop are checked. Advantageously, a further inert material layer can be introduced in the same way as the uppermost layer, and the fill height and, if appropriate, the pressure drop can be checked.

Finally, in a further process step, the pressure drops over all tubes are measured, the mean of the pressure drops and the deviations from the mean are determined, and, in the event of deviations exceeding a fixed target value, the appropriate catalyst tubes are emptied partly or fully and refilled.

The quality of the filling can preferably be checked by means of a pressure drop measurement. All measurements are recorded in a database, and so the reactor state is 100% documented.

As soon as the catalyst is spent, i.e. the active material is deactivated, it has to be removed from the catalyst tubes. When the shaped catalyst bodies are still free-flowing, i.e. have not formed lumps, they can be disposed of directly into a waste vessel by means of a vacuum suction system which is arranged so as to be mobile as a tool on the tool carrier device. The vacuum conveying system is equipped with a suction tube which is preferably introduced under force control down to the lower end of each catalyst tube.

When the shaped catalyst bodies have caked, formed lumps or become compressed together, they have to be drilled out or milled out, in which case the loose material can be sucked out upward. For this purpose, a drilling head or a milling head is arranged so as to be mobile as the tool on the tool carrier arm, and is introduced under force control down to the lower end of each catalyst tube.

It should be ensured that the distance between the external diameter of the drill bit and the inner wall of the catalyst tubes is sufficiently great, more particularly is not less than half the mean particle diameter of the shaped catalyst bodies, in order that the catalyst particles do not become jammed and damage the inner wall of the catalyst tubes.

The drilling head has a drill bit preferably made of a hard metal material, which is hollow and attached to a vacuum suction system. The drill bit is preferably moved in a vibrating motion of especially 180° about the tube axis in the cross-sectional plane of the tubes, preferably with a torque limit in order to prevent damage to the tube, preferably advancing under force control.

After removing the shaped catalyst bodies down to a residual fill height of from about 3 to 5 cm, in a further process step, the retaining device is pushed mechanically out of each catalyst tube.

In a further process step, the inner walls of the catalyst tubes are subsequently cleaned analogously to the first cleaning, i.e. especially by means of high pressure or mechanically, by means of a brush.

A next process step is a material test of the catalyst tube cleaned after emptying of the shaped catalyst bodies, especially by means of eddy currents, in order to identify material damage and if appropriate to exchange damaged catalyst tubes. More particularly, the current state of each catalyst tube is compared to its state immediately after the zero measurement.

For the monitoring of the course of the process, it is advantageous to provide covering stoppers for the upper end of the catalyst tubes, which differ in color, specifically by virtue of covering stoppers for catalyst tubes in which a particular process step is yet to be carried out having a first color, and covering stoppers for catalyst tubes in which the same process step has already been performed having a second color, and all catalyst tubes apart from the catalyst tube which has been accessed by a tool to perform a process step always being covered.

In a further advantageous configuration, a device for removal of dust by suction can be provided in the region of the upper tube plate, above the upper orifices of the catalyst tubes.

The invention also provides for the use of an above-described process or of an above-described apparatus for automatic catalyst exchange for preparing (meth)acrolein, (meth)acrylic acid, phthalic anhydride, maleic anhydride, glyoxal, ethylene oxide or phosgene.

The invention is illustrated in detail hereinafter with reference to a drawing.

The individual figures show:

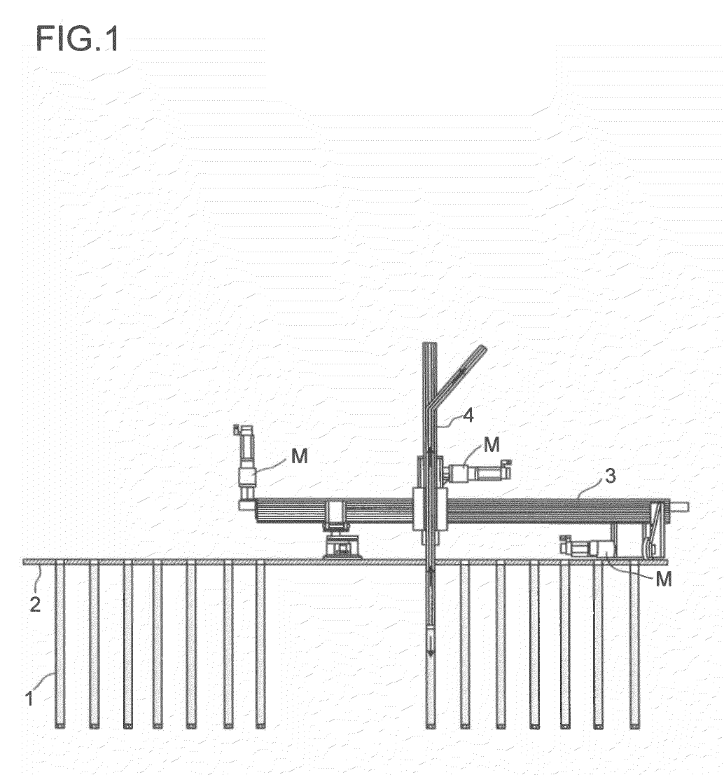

FIG. 1 the schematic diagram of an inventive apparatus for performing the process step of removing spent catalyst material by suction,

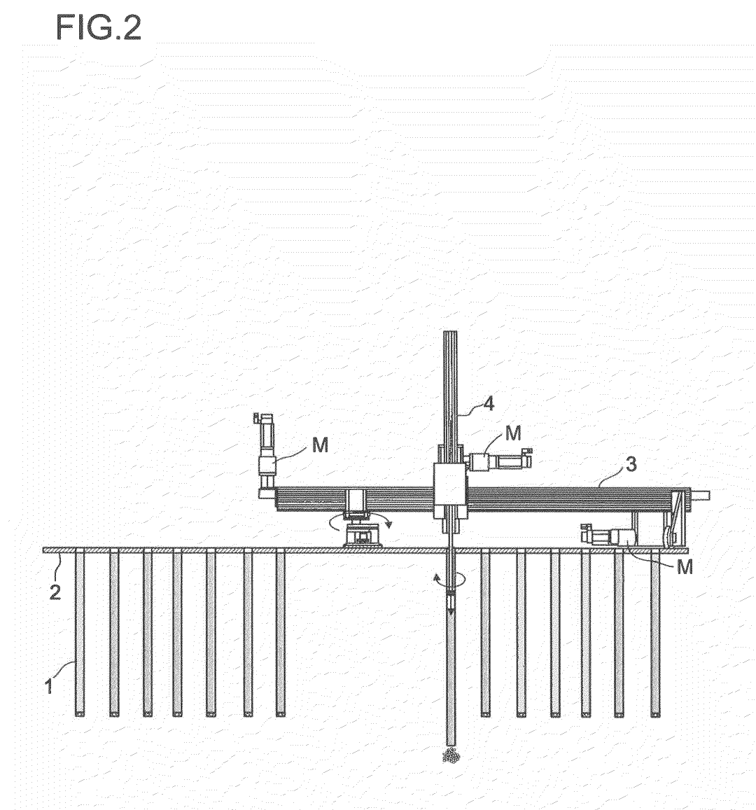

FIG. 2 the schematic diagram of an inventive apparatus for performing the process step of removing spent catalyst material,

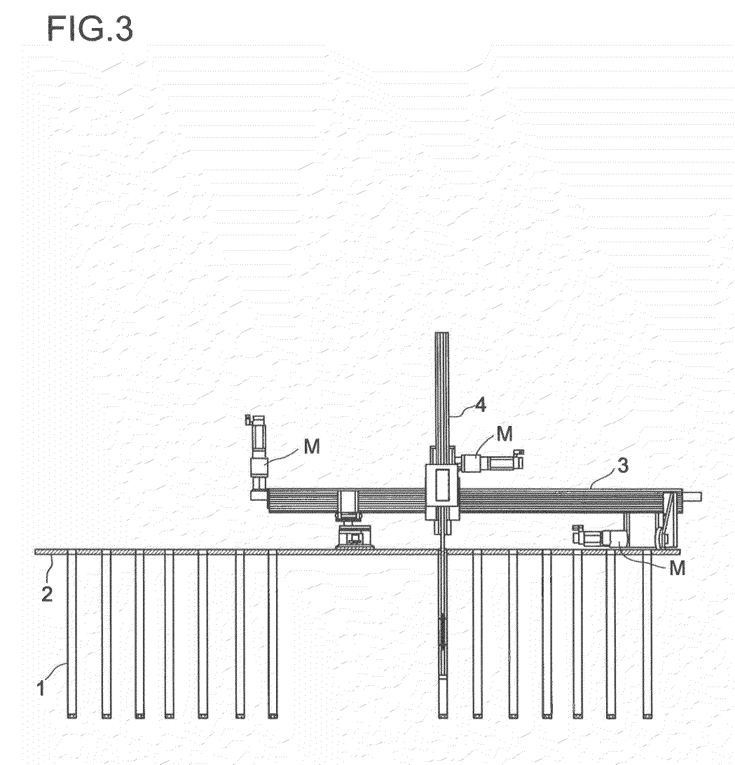

FIG. 3 the schematic diagram of an inventive apparatus for performing the process step for cleaning the emptied catalyst tubes and

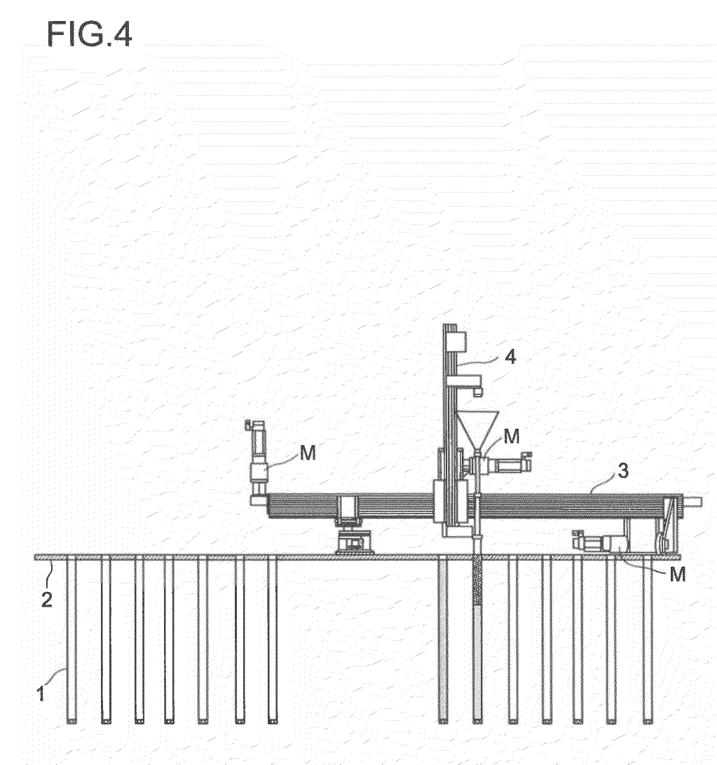

FIG. 4 the schematic diagram of an inventive apparatus for performing the process step of filling the catalyst tubes with shaped catalyst bodies.

The schematic diagram in FIG. 1 shows catalyst tubes 1, which are arranged in parallel to one another in the longitudinal direction of a reactor which is not shown, and are welded into an upper tube plate 2.

In a plane above the upper tube plate 2 and parallel thereto, a tool carrier arm 3 is arranged so as to be mobile and is equipped with a tool 4.

The tool carrier arm 3 is, as shown by way of example in the figure, moved by means of two servomotors M in a plane parallel to the upper tube plate 2.

On the tool carrier arm 3 is in each case arranged, so as to be exchangeable, a tool 4 which is movable by means of a servomotor M vertically in the direction of the longitudinal axis of the reactor and along the tool carrier arm 3.

FIGS. 1 to 4 differ merely by the tools 4, which are selected suitably for the particular process step: in FIG. 1, a vacuum conveying system is provided for removal of spent catalyst material by suction as the tool 4;

in FIG. 2, a drilling head with a core bit is provided to force out catalyst particles which have formed lumps,
in FIG. 3, by way of example, a high-pressure cleaner for cleaning the emptied catalyst tubes 2 is provided, and
in FIG. 4, the tool 4 provided for the process step of filling the catalyst tubes 2 with catalyst particles is a supply tube with defined volume for volumetric release.

1. An apparatus for automatic catalyst exchange in a plurality of process steps in the catalyst tubes of a reactor with a bundle of catalyst tubes, which are welded in or rolled in between an upper tube plate and a lower tube plate in longitudinal direction of the reactor, and welded tight,
comprising a tool carrier device, which is arranged so as to be mobile in a plane above and parallel to the upper tube plate, and which can be equipped with an exchangeable tool for each process step, the exchangeable tool being mobile together with the tool carrier device in the plane above and parallel to the upper tube plate and also vertically, in the direction of the longitudinal axis of the reactor, and being able to access each individual catalyst tube with control by means of a control unit, the position of the longitudinal axis of each individual catalyst tube being measured or calculated and recorded in the control unit in a position database.

2. The apparatus according to claim 1, wherein the tool carrier device comprises a tool carrier arm.

3. The apparatus according to claim 2, wherein the tool carrier device comprises one or more further tool carrier arms(s), with tools arranged exchangeably thereon, said tools being provided on each tool carrier arm for the same process step or for different process steps in each case.

4. The apparatus according to claim 1, wherein covering stoppers for the upper end of the catalyst tubes are provided and differ in color, by virtue of covering stoppers for catalyst tubes in which a particular process step is yet to be carried out having a first color, and covering stoppers for catalyst tubes in which the same process step has already been performed having a second color, and all catalyst tubes apart from the catalyst tube which has been accessed by a tool to perform a process step always being covered.

5. The apparatus according to claim 1, wherein a device for removal of dust by suction is provided in the region of the upper tube plate, above the upper orifices of the catalyst tubes.

6. The apparatus according to claim 1, wherein the reactor comprises from 100 to 40 000 catalyst tubes.

7. A process for automatic catalyst exchange in a plurality of process steps in the catalyst tubes of a reactor with a bundle of catalyst tubes, which are welded in or rolled in between an upper tube plate and a lower tube plate in longitudinal direction of the reactor, and welded tight,
the position of the longitudinal axis of each individual catalyst tube first being determined, especially by means of a 3D laser scanner, or calculated, and recorded in a position database of a control unit,
and then the control unit being used to access each individual catalyst tube of the bundle of catalyst tubes with a tool in each case to perform each individual process step for automatic catalyst exchange, the tool being arranged so as to be exchangeable on a tool carrier device which is arranged so as to be mobile in a plane above and parallel to the upper tube plate, and the tool together with the tool carrier device being moved in the plane above and parallel to the upper tube plate, and also vertically in the direction of the longitudinal axis of the reactor, while each individual process step for automatic catalyst exchange is performed in each case.

8. The process according to claim 7, wherein the process step for automatic catalyst exchange is a first cleaning of catalyst tubes, which have yet to be put into operation, especially high-pressure cleaning or mechanical cleaning.

9. The process according to claim 7, wherein the process step is a material test, especially by means of eddy currents, in order to identify material damage to the catalyst tubes and to exchange damaged catalyst tubes if required.

10. The process according to claim 7, wherein the process step is a check, especially a visual check as to whether a catalyst retaining device is present at the lower end of each catalyst tube.

11. The process according to claim 7, wherein the process step is the filling of each catalyst tube with one or more layers of shaped catalyst bodies and/or with one or more layers of inert material, by means of a load cell with strain gauges, or volumetrically.

12. The process according to claim 7, wherein the process step is the checking of the fill height and/or of the pressure drop after filling with the one layer or any of the plurality of layers of shaped catalyst bodies and/or of inert material.

13. The process according to claim 7, wherein two or more layers of shaped catalyst bodies and/or two or more layers of inert material are introduced into each catalyst tube, the pressure drops over all catalyst tubes are measured, a mean for the pressure drop is determined and the deviation from the mean is calculated for each individual catalyst tube, and, in the event of deviations exceeding a preset, permissible limit, the appropriate catalyst tube is marked, emptied partly or fully and refilled.

14. The process according to claim 7, wherein the process step is the emptying of the spent shaped catalyst bodies from the catalyst tubes or, when the shaped catalyst bodies are no longer free-flowing, by drilling by means of a drilling head or a milling head.

15. The process according to claim 7, wherein the process step is the emptying of the catalyst tubes of shaped catalyst bodies and then the catalyst retaining device is pushed out of each catalyst tube by means of a suitable tool.

16. The process according to claim 7, wherein the process step is the cleaning of the inner walls of the catalyst tubes after emptying of the spent shaped catalyst bodies.

17. The process according to claim 7, wherein the process step is a material test of the catalyst tube cleaned after emptying of the shaped catalyst bodies, in order to identify material damage and if appropriate to exchange damaged catalyst tubes.

18. A method of using an apparatus according to claim 1 or of a process according to claim 7 for automatic catalyst exchange in the catalyst tubes of a reactor for preparing (meth)acrolein, (meth)acrylic acid, phthalic anhydride, maleic anhydride, glyoxal, ethylene oxide or phosgene.
